(19) United States
(12) Patent Application Publication
(10) Pub. No.: US 2005/0107330 A1
(43) Pub. Date: May 19, 2005

(54) Pharmaceutical composition for the treatment of rhinitis

(76) Inventors: Harald Greve, Dusseldorf (DE); Rainer Greve, Bad Segeberg (DE)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/984,628

(22) Filed: Nov. 8, 2004

(30) Foreign Application Priority Data
Nov. 13, 2003 (DE) DE10353690.6
Dec. 2, 2003 (DE) DE10356248.6

(51) Int. Cl. A61K 31/728; A61K 31/4164

(52) U.S. Cl. 514/54; 514/389

(57) ABSTRACT

The present invention relates to a pharmaceutical composition suitable for the prophylactic and/or curative topical treatment of rhinitis, said composition containing in combination and in each case in pharmaceutically efficacious amounts: (a) at least one sympathomimetic suitable for topical application and having vasoconstrictor action and/or detumescent action on the mucous membrane or its physiologically acceptable salts or derivatives; and (b) at least one preferably acidic glycosaminoglycan or its physiologically acceptable salts or derivatives, in particular hyaluronic acid or its salts. On account of a synergistic combined action of the glycosaminoglycan with the sympathomimetic, drying out and inflammatory irritation of the nasal mucous membranes caused by the sympathomimetic on topical application of the pharmaceutical composition are avoided and a detumescence of the mucous membrane in the nasal cavity and an inhibition of secretion are simultaneously achieved, which leads to an efficient alleviation of rhinitis.

### CROSS-REFERENCE TO RELATED APPLICATION

The present application claims the benefit of German Patent Application No. DE 10353690.6 dated Nov. 13, 2003 and German Patent Application No. DE 10356248.6 dated Dec. 2, 2003.

The present invention relates to a pharmaceutical composition based on a sympathomimetic having vasoconstrictor action or detumescent action on the mucous membrane in combination with a preferably acidic glycosaminoglycan, in particular hyaluronic acid or its salts, which is suitable for the prophylactic and/or curative topical treatment of rhinitis. Furthermore, the present invention relates to the use of said pharmaceutical composition for the prophylactic and/or curative topical treatment of rhinitis of all types, in particular acute rhinitis as well as chronic rhinitis.

For the treatment of rhinitis, a large number of sympathomimetics having vasoconstrictor properties or detumescent properties on the mucous membrane are available which lead to detumescence of the nasal mucous membrane on topical or local application to the nose. Repeated application of these substances, however, often leads to a drying out of the nasal mucous membranes, combined with inflammatory irritation. These side effects not unusually lead to an increased danger of infection, since the mucous membranes in the dried out and inflamed state can no longer maintain their protective and filter functions to the full extent, such that pathogens can pass into the airways unhindered.

In order to counteract these generally known side effects of sympathomimetics, it is proposed in the German patent applications DE 195 41 919 A1 and DE 195 49 421 A1 going back to the applicant itself to add efficacious amounts of pantothenol or pantothenic acid to pharmaceutical preparations for the treatment of rhinitis based on sympathomimetics having vasoconstrictor action or detumescent action on the mucous membrane. Pantothenol or pantothenic acid in combination with a sympathomimetic suitable for topical application counteracts a drying out and thus an inflammatory irritation of the nasal mucous membranes.

The present invention is now based on the object of making available a pharmaceutical composition which is suitable for the topical treatment of rhinitis and in particular at least largely avoids or at least diminishes the disadvantages of the prior art previously outlined.

A further object of the present invention lies in making available a pharmaceutical composition based on sympathomimetics having vasoconstrictor properties or properties having a detumescent action on the mucous membrane which avoids drying out and inflammatory irritation of the nasal mucous membranes. In particular, an alternative to the compositions according to DE 195 41 919 A1 and DE 195 49 421 A1 known from the prior art and going back to the applicant itself should be made available here or the compositions described there should be developed further.

The applicant has now surprisingly found out that the problem outlined before can be solved by formulating a pharmaceutical preparation based on a sympathomimetic suitable for topical application having vasoconstrictor action or detumescent action on the mucous membrane or its pharmaceutically acceptable salts in combination with at least one preferably acidic glycosaminoglycan, preferably hyaluronic acid or its physiologically acceptable salts.

The present invention thus relates to a pharmaceutical composition which is suitable for the prophylactic and/or curative topical treatment of rhinitis, the pharmaceutical composition containing in combination and in each case in pharmaceutically efficacious amounts
(a) at least one sympathomimetic suitable for topical application and having vasoconstrictor action and/or detumescent action on the mucous membrane or its physiologically acceptable salts or derivatives; and
(b) at least one preferably acidic glycosaminoglycan or its physiologically acceptable salts or derivatives, in particular hyaluronic acid or its salts.

Surprisingly, the preferably acidic glycosaminoglycan, in particular hyaluronic acid or its physiologically tolerable or physiologically acceptable salts, counteracts a drying out of the nasal mucous membranes caused by the application of the sympathomimetic and in this way prevents inflammatory irritation of the nasal mucous membranes.

According to the invention, hyaluronic acid is particularly preferably employed as the glycosaminoglycan, in particular in the form of its physiologically tolerable salts, preferably in the form of its sodium salt. Hyaluronic acid (hyaluronan) is an acidic glycosaminoglycan (mucopolysaccharide) of biological origin, which has been isolated for the first time from the vitreous body of cows' eyes and which also occurs in the synovial fluid of the joints and in the skin, where it forms more than 50% of the skin matrix. Hyaluronic acid and its derivatives, in particular its salts, are distinguished by a high waterbinding power; hyaluronic acid is a high molecular weight compound forming highly viscous, aqueous solutions, for which, depending on origin, workup and determination methods, molar masses of between 50,000 and a number of million are indicated. For further details, reference can be made, for example, to Römpp Chemielexikon (Römpp's Chemical Encyclopaedia), 10th Edition, Volume 3, page 1820, Keyword: “Hyaluronic acid”, Georg Thieme Verlag Stuttgart/New York, 1997.

It is completely surprising that the use of glycosaminoglycans, preferably acidic glycosaminoglycans, in particular hyaluronic acid or its salts, in combination with a sympathomimetic suitable for topical application and having vasoconstrictor action or detumescent action on the mucous membrane or its physiologically acceptable salts is much better suited for the treatment of rhinitis than known monopreparations, which only contain the sympathomimetic, and that, by means of a synergistic effect of the combined action of the glycosaminoglycan with the sympathomimetic, drying out and inflammatory irritation of the nasal mucous membranes are avoided in the topical or local application according to the invention of the pharmaceutical composition. Unexpectedly, in studies by the applicant, as a result of the synergistic combined action of the sympathomimetic with the glycosaminoglycan, a markedly accelerated process of recovery from the rhinitis, in particular of acute rhinitis, was observed with topical application of the pharmaceutical composition according to the invention.

The amount of glycosaminoglycan or its physiologically acceptable salts or derivatives, in particular hyaluronic acid or its physiologically acceptable salts, in the pharmaceutical composition according to the invention can vary within wide ranges. In general, the glycosaminoglycan or its physiologically acceptable salts or derivatives, in particular hyaluronic acid or its physiologically acceptable salts, is present in amounts from 0.01 to 5% by weight, in particular 0.05 to 1% by weight, preferably 0.05 to 0.25% by weight, based on the pharmaceutical composition. On significantly falling short of the lower limit of 0.01% by weight, in general too low an action is achieved, whereas in the case of excessively large concentrations markedly above 5% by weight, in general an excessively great thickening of the pharmaceutical composition according to the invention is observed. Nevertheless, if appropriate it may be necessary to depart from the amounts mentioned if the therapeutic conditions require this.

As sympathomimetics having vasoconstrictor action or detumescent action on the mucous membrane, according to the invention all sympathomimetics known from the prior art for these purposes can be possible, in particular α-sympathomimetics suitable for topical application, preferably α-sympathomimetics having a 2-imidazoline structure, or their physiologically acceptable salts.

As is known, sympathomimetically active imidazoline derivatives cause contraction of the vessels, in particular in the mucous membrane, and are therefore given locally for the detumescence of the mucous membrane in the nasal cavity. The inhibition of secretion and detumescence of the mucous membranes lead to an alleviation of the rhinitis. Such α-sympathomimetics based on imidazoline derivatives, such as, for example, oxymetazoline, xylometazoline, tramazoline, tetryzoline and naphazoline, directly stimulate the α-adrenergic receptors of the sympathetic nervous system, but have less or no action on β-adrenergic receptors. The intranasal administration of such α-sympathomimetics in general leads to the constriction of dilated arterioles and thus to the normalization of the increased mucous membrane circulation, to the reduction of the formation of oedema and to the improvement of nasal ventilation. Owing to the ventilation of the nasal sinuses and the eustachian tube, the danger of complications, e.g. as a result of a secretion blockage, decreases. After intranasal administration of α-sympathomimetics, local vasoconstriction usually occurs within short periods of time, for example within 5 to 10 minutes, and in general persists for a number of hours.

As a result of a “rebound effect”, after repeated application of sympathomimetics medicinally related rhinitis with drying out and inflammatory irritation of the nasal mucous membrane can occur, such that the varied therapeutic application possibilities of the sympathomimetics are largely restricted. However, it has now surprisingly been found that on local application of glycosaminoglycans, preferably acid glycosaminoglycans, in particular hyaluronic acid or its salts, in combination with sympathomimetics to the nasal mucous membrane even after short treatment times a manifest recovery result is recorded and inflammatory irritation and drying out of the nasal mucous membranes are avoided.

According to the invention, preferred examples of α-sympathomimetics suitable for topical application having a 2-imidazoline structure or their physiologically acceptable salts are oxymetazoline, xylometazoline, tramazoline, tetryzoline and naphazoline, and their physiologically acceptable salts, in particular their hydrochlorides.

Oxymetazoline and xylometazoline and their physiologically acceptable salts, in particular their hydrochlorides, are particularly preferred according to the invention.

Suitable sympathomimetics which can be used according to the invention are, however, also ephedrine and ephedrine derivatives, such as pseudoephedrine, norephedrine, norpseudoephedrine, N-methylephedrine and N-methylpseudoephedrine. According to the invention, (−)-ephedrine or (1R,2S)-2-methylamino-1-phenyl-1-propanol is very particularly preferred. Ephedrine and ephedrine derivatives are alkaloids from Ephedra species. They are indirectly acting sympathomimetics having vasoconstrictor action. Naturally occurring (−)-ephedrine or (1R,2S)-2-methylamino-1-phenyl-1-propanol is particularly active. The compound (1S,2R)-ephedrine enantiomeric to this shows only approximately one third of the pharmacological action of the natural form. The two diastereomers of ephedrine and its synthetic enantiomer are called pseudoephedrines. For further details regarding ephedrine and ephedrine derivatives, reference can be made to Römpp Chemielexikon (Römpp's Chemical Encyclopaedia), 10th Edition, Volume 2, pages 1181/1182, Keyword: “Ephedrine”, Georg Thieme Verlag Stuttgart/New York, 1997 and the literature referred to there, as well as to ROCHE-Lexikon Medizin (ROCHE Medical Encyclopaedia), 3rd Edition, page 478, Keyword: “Ephedrine”, Verlag Urban & Schwarzenberg, the contents of the aforementioned literature hereby being included completely by way of reference.

The amount of sympathomimetic in the pharmaceutical composition according to the invention can vary within wide ranges. In general, the sympathomimetic is present in amounts from 0.001 to 1% by weight, in particular 0.01 to 0.1% by weight, preferably 0.01 to 0.05% by weight, based on the pharmaceutical composition. If the lower limit of 0.001% by weight is significantly fallen short of, in general too low an action is achieved, whereas excessively large concentrations markedly above 1% by weight in general lead to no noticeable increase in the therapeutic effect. Nevertheless, if appropriate it may be necessary to depart from the amounts mentioned if the therapeutic conditions require this.

Particularly good results are surprisingly achieved if additionally pantothenol or its physiologically acceptable derivatives, in particular esters, (e.g. esters with pharmaceutically acceptable carboxylic acids) and/or pantothenic acid or its physiologically acceptable salts are moreover also added to the pharmaceutical composition as further components—in combination with the sympathomimetic and the glycosaminoglycan. D(+)-Pantothenol(dexpanthenol) or its physiologically acceptable derivatives, in particular its esters, are particularly preferred.

The invention thus relates—according to a preferred embodiment—to a pharmaceutical composition suitable for the prophylactic and/or curative topical treatment of rhinitis, which in combination and in each case in pharmaceutically efficacious amounts contains
(a) at least one sympathomimetic suitable for topical application and having vasoconstrictor action and/or detumescent action on the mucous membrane or its physiologically acceptable salts or derivatives;
(b) at least one preferably acidic glycosaminoglycan or its physiologically acceptable salts or derivatives, in particular hyaluronic acid or its salts; and
(c) pantothenol or its derivatives, in particular esters, and/or pantothenic acid or its physiologically acceptable salts.

It was not to be expected that pantothenol or its derivatives and/or pantothenic acid or its physiologically acceptable salts leads, in synergistic combination with the sympathomimetic and the glycosaminoglycan, preferably hyaluronic acid or its salts, to a particularly improved pharmaceutical composition for the treatment of rhinitis, because a composition of this type, in addition to a vasoconstrictor action or detumescent action on the mucous membrane, counteracts any drying out with accompanying inflammatory irritation of the nasal mucous membranes particularly well or avoids this.

The ratios of the different components (a), (b) and (c) present in the pharmaceutical composition of the present invention may vary in broad ranges. Especially, the pharmaceutical composition according to the present invention may contain the components (a) and (b) in weight-related quantitative ratios of (a):(b) in the range from 100:1 to 1:5,000 and/or the components (a) and (c) in weight-related quantitative ratios of (a):(c) in the range from 100:1 to 1:15,000 and/or the components (b) and (c) in weight-related quantitative ratios of (b):(c) in the range from 500:1 to 1:1,500.

The amount of pantothenol or its derivatives and/or pantothenic acid or its physiologically acceptable salts in the pharmaceutical composition according to the invention can vary within wide ranges. In general, the pharmaceutical composition according to the invention contains pantothenol or its derivatives, in particular esters, and/or pantothenic acid or its physiologically acceptable salts in amounts of altogether 0.01 to 15% by weight, in particular 0.1 to 10% by weight, preferably 0.2 to 5% by weight, based on the pharmaceutical composition. Nevertheless, if appropriate it may be necessary to depart from the amounts mentioned, if the therapeutic conditions require this.

A preferred pharmaceutical composition according to the invention, which is particularly suitable for the prophylactic and/or curative topical treatment of rhinitis, contains—in combination and in each case based on the pharmaceutical composition
(a) 0.001 to 1% by weight, in particular 0.01 to 0.1% by weight, preferably 0.01 to 0.05% by weight, of at least one sympathomimetic suitable for topical application and having vasoconstrictor action and/or detumescent action on the mucous membrane or its physiologically acceptable salts or derivatives;
(b) 0.01 to 5% by weight, in particular 0.05 to 1% by weight, preferably 0.05 to 0.25% by weight, of at least one preferably acidic glycosaminoglycan or its physiologically acceptable salts or derivatives, in particular hyaluronic acid or its salts; and
(c) optionally 0.01 to 15% by weight, in particular 0.1 to 10% by weight, preferably 0.2 to 5% by weight, of pantothenol or its derivatives, in particular esters, and/or pantothenic acid or its physiologically acceptable salts.

A particularly preferred pharmaceutical composition according to the invention, which is particularly suitable for the prophylactic and/or curative topical treatment of rhinitis, contains—in combination and in each case based on the pharmaceutical composition
(a) 0.001 to 1% by weight, in particular 0.01 to 0.1% by weight, preferably 0.01 to 0.05% by weight, of oxymetazoline and/or xylometazoline, preferably in the form of their physiologically acceptable salts, in particular in hydrochloride form;
(b) 0.01 to 5% by weight, in particular 0.05 to 1% by weight, preferably 0.05 to 0.25% by weight, of hyaluronic acid, preferably in the form of its physiologically acceptable salts, in particular as the sodium salt; and
(c) optionally 0.01 to 15% by weight, in particular 0.1 to 10% by weight, preferably 0.2 to 5% by weight, of pantothenol or its esters and/or pantothenic acid or its physiologically acceptable salts.

The pharmaceutical composition according to the invention can have a liquid or viscous to semisolid consistency. For example, the pharmaceutical composition according to the invention can be present, for example, as an ointment, cream or gel for introduction into the nose or as a solution or dispersion for dripping or spraying into the nose.

Suitable vehicles for liquid administration forms are, in particular, aqueous systems with or without addition of glycerol, sorbitol or other polyols. Suitable vehicles for viscous or semisolid pharmaceutical preparations, such as, for example, ointments, creams or gels, are, for example, paraffin hydrocarbons, petroleum jelly, wool wax products and other pharmaceutically utilizable, viscosity-increasing basic substances, in the case of hydrophilic gels, for example, water, glycerol or sorbitol, which are gelled using suitable substances that swell, such as, for example, polyacrylic acid, cellulose derivatives, starch or tragacanth (traganth).

Besides the active substances and vehicles and emulsifiers optionally present, the pharmaceutical composition according to the invention can also contain other pharmaceutical excipients and/or additives which are harmless and pharmaceutically compatible in relation to the active compounds, for instance fillers, extenders, binding agents, wetting agents, stabilizing agents, colourants, buffers and aromatic substances. Furthermore, microbiologically active chemical compounds, such as, for example, preservatives or antiseptics for improving the microbial stability, can be present in the pharmaceutical composition according to the invention in pharmaceutically customary concentrations. Moreover, the pharmaceutical composition according to the invention can also additionally contain one or more other pharmacologically active substances.

The pharmaceutical composition according to the invention can be prepared in a manner known per se. This is carried out, for example, by mixing or dissolving the active compounds in pharmacologically efficacious concentrations, the excipients and/or additives and, optionally, the further pharmacologically active substances in the intended carrier medium.

For the demonstration of the action, in particular of the synergistic action, of the pharmaceutical composition according to the invention, in each case one side of the nose was treated in experiments with a sympathomimetic on its own and the other side of the nose with a pharmaceutical composition according to the invention. In these experiments, ongoing rhinoscopy in the case of the side of the nose treated with the pharmaceutical composition according to the invention showed a marked improvement compared with the side of the nose treated with the sympathomimetic on its own. Moreover, it was seen that the glycosaminoglycan alleviated or completely prevented the irritant action of the sympathomimetic and drying out of the mucous membranes and consequently infectious irritation. This effect was increased even further in a composition according to the invention which additionally also contained pantothenol or pantothenic acid.

The present invention further relates to the use of the pharmaceutical composition according to the present invention for the prophylactic and/or curative topical treatment of rhinitis or for the production of a medicament for the prophylactic and/or curative topical treatment of rhinitis.

The pharmaceutical composition according to the invention is suitable for the treatment of rhinitis of all types, namely acute rhinitis as well as chronic rhinitis. By way of example, in this connection rhinitis acuta, rhinitis allergica, rhinitis atrophicans, rhinitis hyperplastica or hypertrophicans, rhinitis mutilans, rhinitis nervosa or vasomotorica, rhinitis pseudomembranacea and rhinitis sicca may be mentioned. The pharmaceutical composition according to the invention is particularly suitable for the treatment of acute rhinitis. However, chronic rhinitis can also be treated well using the pharmaceutical composition according to the invention, in particular on account of avoidance of drying out and inflammatory irritation of the nasal mucous membranes.

For this purpose, the pharmaceutical composition according to the invention is in general administered intranasally, in particular a number of times daily. The pharmaceutical composition according to the invention leads to a detumescence or vasoconstriction of the affected nasal mucous membranes and in this way to a normalization of the increased mucous membrane circulation and to a reduction of the formation of oedema, accompanied by an improvement in nasal ventilation, in particular to improved ventilation of the nasal sinuses and the eustachian tubes, and thus to prevention of a secretion blockage. At the same time, drying out and inflammatory irritation of the nasal mucous membranes are prevented.

Further embodiments, modifications and variations and advantages of the present invention are discernible and realizable without any problems for the person skilled in the art on reading the description, without leaving the scope of the present invention.

The following working examples serve only to illustrate the present invention, without, however, restricting it hereto.

### WORKING EXAMPLES

### Example 1

### (According to the Invention)

For the preparation of 100 g of a clear aqueous solution, 50 g of purified water are introduced into a 250 ml beaker. 0.25 g of hyaluronic acid in the form of its sodium salt is then introduced with stirring as an aqueous solution. 0.02 g of benzalkonium chloride is then added to the solution as a preservative and likewise dissolved with stirring. 0.05 g of oxymetazoline in the form of the hydrochloride is then added. The whole is made up to the final weight of 100.0 g with further water and stirred until homogeneous. The homogeneous solution is then filled, optionally after filtration through a neutral cellulose filter, into narrow-necked flasks of 10 or 20 ml made of brown glass, which are alternatively equipped with a dropping pipette or a spraymetering pump. For the administration of the solution according to the invention, one or more drops or one or more puffs of spray are administered to each nostril a number of times daily.

### Example 2

### (According to the Invention)

Example 1 is repeated, but with the exception that 5 g of water are replaced by 5 g of dexpanthenol.

### Example 3

### (Not According to the Invention)

Example 1 is repeated, but with the exception that the hyaluronic acid content is completely replaced by water. A solution not according to the invention results, which only contains oxymetazoline hydrochloride as active compound.

### Example 4

Ten patients with acute rhinitis were treated both with the solution not according to the invention as in Example 3 and either with the solution according to the invention as in Example 1 or Example 2, five patients being treated with the solutions as in Examples 1 and 3 and the other five patients being treated with the solutions as in Examples 2 and 3. In this case, one side of the nose was in each case treated with the solution not according to the invention containing oxymetazoline hydrochloride on its own as in Example 3 and the other side of the nose was treated with the preparation according to the invention as in Example 1 or 2, and the treated patients were examined at regular intervals by means of rhinoscopy.

In the case of all solutions a vasoconstrictor effect occurred, caused by the oxymetazoline. The observation that the irritant action emanating from oxymetazoline did not occur on administration of the solutions according to the invention as in Examples 1 and 2, wherefrom greater compliance of the patients results, is remarkable and surprising, whereas in the case of the solution not according to the invention as in Example 3 drying out and inflammatory irritation occurred. The detumescent effect on the mucous membrane proved to be significantly more marked than expected after treatment with the combinations according to the invention as in Examples 1 and 2: the effect lasted longer after treatment with the solutions according to the invention as in Example 1 and 2, and at the same time a markedly better action was achieved. In this case, it was seen that in the case of intranasal administration both in the case of the binary active compound composition as in Example 1 and in the case of the ternary active compound composition as in Example 2 a markedly improved effect occurred compared with the monopreparation, which was seen in that in the case of the compositions according to the invention no drying out of the nasal mucous membranes occurred, whereas in the case of monotherapy with oxymetazoline on its own drying out of the mucous membrane with accompanying inflammatory irritation occurred.

The rhinoscopic examinations show that the application of the ternary composition according to the invention as in Example 2 compared with the binary mixture as in Example 1 brings about additionally increased success, since the rhinitis and inflammation abated more rapidly.

On the basis of the previously described results, the application of the monosubstance was usually terminated after a few days in order to be continued with markedly improved effect with the combinations according to the invention.

The results of the experiments show a marked synergism of the actions of the α-sympathomimetic oxymetazoline on the one hand and of hyaluronic acid on the other hand, which can additionally be increased by addition of pantothenol; the pharmaceutical compositions according to the invention lead to an impressive improvement in the treatment of acute rhinitis which goes markedly beyond the extent of the individual active compounds, which supports the synergistic effect.

1. A pharmaceutical composition for the prophylactic or curative topical treatment of rhinitis, said composition comprising in combination and in each case in pharmaceutically efficacious amounts:
(a) at least one sympathomimetic suitable for topical application and having vasoconstrictor action or detumescent action on the mucous membrane or its physiologically acceptable salts or derivatives; and
(b) at least one acidic glycosaminoglycan or its physiologically acceptable salts or derivatives.

2. The pharmaceutical composition according to claim 1, wherein the sympathomimetic is an α-sympathomimetic having a 2-imidazoline structure and being selected from the group consisting of oxymetazoline, xylometazoline, tramazoline, tetryzoline and naphazoline, and their physiologically acceptable salts or derivatives.

3. The pharmaceutical composition according to claim 1, wherein the sympathomimetic is one of oxymetazoline hydrochloride or xylometazoline hydrochloride.

4. The pharmaceutical composition according to claim 1, wherein the sympathomimetic is present in amounts from 0.001 to 1% by weight, based on the pharmaceutical composition.

5. The pharmaceutical composition according to claim 1, wherein the glycosaminoglycan is hyaluronic acid or its physiologically acceptable salts.

6. The pharmaceutical composition according to claim 5, wherein the physiologically acceptable salt of the hyaluronic acid is the sodium salt of hyaluronic acid.

7. The pharmaceutical composition according to claim 1, wherein the glycosaminoglycan or its physiologically acceptable salts or derivatives is present in amounts from 0.01 to 5% by weight, based on the pharmaceutical composition.

8. The pharmaceutical composition according to claim 1, wherein the pharmaceutical composition further contains (c) a compound selected from the group consisting of: pantothenol, pantothenol ester, pantothenic acid and physiologically acceptable salts of pantothenic acid.

9. The pharmaceutical composition according to claim 8, wherein the pharmaceutical composition contains (c) the compound selected from the group consisting of: pantothenol, pantothenol ester, pantothenic acid and physiologically acceptable salts of pantothenic acid in amounts altogether from 0.01 to 15% by weight, based on the pharmaceutical composition.

10. The pharmaceutical composition according to claim 8, wherein the pharmaceutical composition contains:
the components (a) and (b) in weight-related quantitative ratios of (a):(b) in the range from 100:1 to 1:5,000;
the components (a) and (c) in weight-related quantitative ratios of (a):(c) in the range from 100:1 to 1:15,000; and
the components (b) and (c) in weight-related quantitative ratios of (b):(c) in the range from 500:1 to 1:1,500.

11. The pharmaceutical composition according to claim 1, wherein the composition is formulated as a solution, a dispersion a paste as a spray, an ointment, a cream or a gel.

12. A pharmaceutical composition for the prophylactic or curative topical treatment of rhinitis, said composition comprising in combination and in each case in pharmaceutically efficacious amounts:
(a) at least one sympathomimetic suitable for topical application and having vasoconstrictor action or detumescent action on the mucous membrane or its physiologically acceptable salts or derivatives;
(b) at least one acidic glycosaminoglycan or its physiologically acceptable salts or derivatives; and
(c) at least one of pantothenol, its derivatives, pantothenic acid, or physiologically acceptable salts of pantothenic acid.

13. The pharmaceutical composition according to claim 12, wherein the sympathomimetic is one of oxymetazoline hydrochloride or xylometazoline hydrochloride.

14. The pharmaceutical composition according to claim 12, wherein the glycosaminoglycan is hyaluronic acid or its physiologically acceptable salts.

15. A pharmaceutical composition for the prophylactic or curative topical treatment of rhinitis, said composition comprising in combination and in each case based on the pharmaceutical composition:
(a) 0.001 to 1% by weight of at least one sympathomimetic suitable for topical application and having vasoconstrictor action or detumescent action on the mucous membrane or its physiologically acceptable salts or derivatives;
(b) 0.01 to 5% by weight of at least one acidic glycosaminoglycan or its physiologically acceptable salts or derivatives; and
(c) optionally 0.01 to 15% by weight of pantothenol or its derivatives and/or pantothenic acid or its physiologically acceptable salts.

16. The pharmaceutical composition according to claim 15, wherein the sympathomimetic is one of oxymetazoline hydrochloride or xylometazoline hydrochloride.

17. The pharmaceutical composition according to claim 15, wherein the glycosaminoglycan is hyaluronic acid or its physiologically acceptable salts.

18. A pharmaceutical composition for the prophylactic or curative topical treatment of rhinitis, comprising in combination and in each case based on the pharmaceutical composition
(a) 0.001 to 1% by weight of at least one sympathomimetic selected from the group consisting of oxymetazoline and xylometazoline;
(b) 0.01 to 5% by weight of hyaluronic acid in the form of its physiologically acceptable salts; and
(c) optionally 0.01 to 15% by weight of at least one of pantothenol or its esters and/or pantothenic acid or its physiologically acceptable salts.

19. A method of treating a human suffering from rhinitis, said method comprising administering an effective amount of the pharmaceutical composition as defined in claim 1 to said human.

20. A method of treating a human suffering from rhinitis, said method comprising administering an effective amount of the pharmaceutical composition as defined in claim 12 to said human.

21. A method of treating a human suffering from rhinitis, said method comprising administering an effective amount of the pharmaceutical composition as defined in claim 15 to said human.

22. A method of treating a human suffering from rhinitis, said method comprising administering an effective amount of the pharmaceutical composition as defined in claim 18 to said human.
